(19) United States
(12) Patent Application Publication
(10) Pub. No.: US 2025/0082610 A1
(43) Pub. Date: Mar. 13, 2025

(54) COMPOSITION, METHOD FOR MICROORGANISM GROWTH SUPPRESSION IN COMPOSITION FOR ORAL USE, AND AGENT FOR POTENTIATION OF MICROORGANISM-GROWTH-SUPPRESSING EFFECT OF QUERCETIN OR GLYCOSIDE THEREOF

(71) Applicant: SUNTORY HOLDINGS LIMITED, Osaka-shi, Osaka (JP)

(72) Inventors: Makoto Katsube, Soraku-gun (JP); Hiroshi Watanabe, Soraku-gun (JP)

(73) Assignee: SUNTORY HOLDINGS LIMITED, Osaka-shi, Osaka (JP)

(21) Appl. No.: 18/728,706

(22) PCT Filed: Jan. 26, 2023
(86) PCT No.: PCT/JP2023/002341

§ 371 Date: Jul. 12, 2024

(30) Foreign Application Priority Data
Jan. 28, 2022 (JP) 2022-011985

(51) Int. Cl. A61K 31/4172; A61K 31/353; A61K 31/7048; A61P 31/04

(52) U.S. Cl. CPC A61K 31/4172; A61K 31/353; A61K 31/7048; A61P 31/04

(57) ABSTRACT

The present invention aims to provide a technique to effectively inhibit microorganism growth in an oral composition and the like. The present invention relates to a composition containing (A) L-ergothioneine or a salt thereof and (B) quercetin or a glycoside thereof.

### TECHNICAL FIELD

The present invention relates to a composition containing L-ergothioneine or a salt thereof and quercetin or a glycoside thereof. The present invention also relates to a method of inhibiting microorganism growth in an oral composition. The present invention further relates to, for example, an agent for enhancing microorganism growth inhibitory action of quercetin or a glycoside thereof.

### BACKGROUND ART

Inhibiting microorganism growth to increase the shelf life is important for oral compositions such as commercial food or beverages and pharmaceutical products. Thus, a naturally derived component that is effective in inhibiting microorganism growth and that is highly safe is useful.

Quercetin is a type of flavonoids and contained as is or in the glycoside form in plants such as onions. Non-Patent Literature 1 describes inhibition of growth of Escherichia coli and Staphylococcus aureus by quercetin.

Ergothioneine is one of the sulfur-containing amino acids. L-ergothioneine is present in nature. L-ergothioneine is known to have useful physiological activities such as antioxidant action. For example, according to Patent Literature 1, the expression level of mRNA in human β-defensin-1 (hBD-1) and human β-defensin-3 (hBD-3) was increased when ergothioneine derived from Pleurotus citrinopileatus was allowed to act on human immortalized keratinocyte cells (HaCaT cells).

### CITATION LIST

### Patent Literature

Patent Literature 1: JP 2012-56914 A

### Non-Patent Literature

Non-Patent Literature 1: Journal of Chemical and Pharmaceutical Research, 2015, 7(12):834-841

### SUMMARY OF INVENTION

### Technical Problem

The present invention aims to provide a technique to effectively inhibit microorganism growth in an oral composition and the like.

### Solution to Problem

As a result of extensive studies to solve the above problem, the present inventors found that L-ergothioneine enhances the microorganism growth inhibitory action of quercetin or a glycoside thereof. Use of L-ergothioneine or a salt thereof and quercetin or a glycoside thereof in combination can effectively inhibit microorganism growth.

Specifically, the present invention relates, but is not limited, to a composition, a method of inhibiting microorganism growth in an oral composition, an agent for enhancing microorganism growth inhibitory action of quercetin or a glycoside thereof, and the like, which are defined below.
(1) A composition containing: (A) L-ergothioneine or a salt thereof; and (B) quercetin or a glycoside thereof.
(2) The composition according to (1) above, wherein the (B) quercetin or a glycoside thereof is at least one selected from the group consisting of isoquercitrin, rutin, enzyme-treated rutin, and quercitrin.
(3) The composition according to (1) or (2) above, wherein a weight ratio of the (A) L-ergothioneine or a salt thereof in terms of L-ergothioneine to the (B) quercetin or a glycoside thereof in terms of isoquercitrin, i.e., (A) in terms of L-ergothioneine/(B) in terms of isoquercitrin, is 0.01 to 1.2.
(4) The composition according to any one of (1) to (3) above, wherein the composition is a composition for inhibiting microorganism growth.
(5) The composition according to (4) above, wherein the composition is a composition for inhibiting microorganism growth in an oral composition.
(6) The composition according to (4) or (5) above, wherein the microorganism is Escherichia coli.
(7) A method of inhibiting microorganism growth in an oral composition, including adding (A) L-ergothioneine or a salt thereof and (B) quercetin or a glycoside thereof to an oral composition.
(8) The method according to (7) above, wherein the (B) quercetin or a glycoside thereof is at least one selected from the group consisting of isoquercitrin, rutin, enzyme-treated rutin, and quercitrin.
(9) The method according to (7) or (8) above, wherein the (A) L-ergothioneine or a salt thereof and the (B) quercetin or a glycoside thereof are added to an oral composition such that a weight ratio of the (A) L-ergothioneine or a salt thereof in terms of L-ergothioneine to the (B) quercetin or a glycoside thereof in terms of isoquercitrin, i.e., (A) in terms of L-ergothioneine/(B) in terms of isoquercitrin, in the oral composition is 0.01 to 1.2.
(10) The method according to any one of (7) to (9) above, wherein the microorganism is Escherichia coli.
(11) An agent for enhancing microorganism growth inhibitory action of quercetin or a glycoside thereof, containing: L-ergothioneine or a salt thereof.

### Advantageous Effects of Invention

The present invention can provide a technique to effectively inhibit microorganism growth in an oral composition and the like.

### BRIEF DESCRIPTION OF DRAWINGS

FIG. 1 is a graph showing a sterilizing effect of L-ergothioneine (test sample 1), isoquercitrin (test sample 2), and a combination of L-ergothioneine and isoquercitrin (test samples 3 and 4).

### DESCRIPTION OF EMBODIMENTS

### (Composition)

The composition of the present invention contains (A) L-ergothioneine or a salt thereof and (B) quercetin or a glycoside thereof.

Quercetin or a glycoside thereof has microorganism growth inhibitory action (antibacterial action). A combination of L-ergothioneine or a salt thereof and quercetin or a glycoside thereof can enhance the microorganism growth inhibitory action of quercetin or a glycoside thereof. Thus, use of L-ergothioneine or a salt thereof and quercetin or a glycoside thereof in combination can effectively inhibit microorganism growth.

L-ergothioneine is one of the amino acids. The salt of L-ergothioneine is not limited as long as it is a pharmacologically acceptable salt or a dietary acceptable salt, and the salt may be either an acid salt or a basic salt. Examples of the acid salt include inorganic acid salts such as hydrochloride, sulfate, nitrate, and phosphate, and organic acid salts such as acetate, citrate, maleate, malate, oxalate, lactate, succinate, fumarate, and propionate. Examples of the basic salt include alkali metal salts such as sodium salt and potassium salt, and alkaline earth metal salts such as calcium salt and magnesium salt.

L-ergothioneine is preferably used as L-ergothioneine or a salt thereof.

Chemically synthesized L-ergothioneine or a salt thereof may be used, or a naturally extracted and purified L-ergothioneine or a salt thereof may be used. L-ergothioneine is abundant in golden/yellow oyster mushrooms (scientific name: Pleurotus cornucopiae var. citrinopileatus) belonging to the genus Pleurotus of the family Pleurotaceae. L-ergothioneine is also present in mushrooms such as common mushrooms (scientific name: Agaricus bisporus) including white button mushrooms, cremini mushrooms, and portabella mushrooms; grey oyster mushrooms (scientific name: Pleurotus ostreatus), shiitake (scientific name: Lentinula edodes), hen-of-the-woods (scientific name: Grifola frondosa), reishi mushrooms (scientific name: Ganoderma lucidum), lion's mane mushrooms (scientific name: Hericium erinaceus), Yanagi-matsutake (scientific name: Agrocybe aegerita), girolles (scientific name: Cantharellus cibarius), porcini mushrooms (scientific name: Boletus edulis), and morel mushrooms (scientific name: Morchella esculenta). When L-ergothioneine is obtained from a natural product, preferably, it is extracted from a golden/yellow oyster mushroom, for example. L-ergothioneine or a salt thereof can also be produced by microbial fermentation. An extract containing L-ergothioneine or a salt thereof produced by microbial fermentation or a purified product of the extract may be used. Extraction and purification from natural products and the like can be performed by known methods. L-ergothioneine or a salt thereof may be in an isolated form.

The amount of L-ergothioneine or a salt thereof in the composition is not limited. For example, the amount in terms of L-ergothioneine is preferably 0.00001 wt % or more, more preferably 0.0001 wt % or more, and may be 50 wt % or less or 10 wt % or less, preferably 0.1 wt % or less, more preferably 0.01 wt % or less. In one embodiment, the amount of L-ergothioneine or a salt thereof in terms of L-ergothioneine in the composition may be 0.00001 to 50 wt % or 0.00001 to 10 wt %, preferably 0.00001 to 0.1 wt %, more preferably 0.0001 to 0.01 wt %. The amount of L-ergothioneine or a salt thereof can be measured by high-performance liquid chromatography (HPLC).

Herein, the expression regarding the amount of L-ergothioneine or a salt thereof in terms of L-ergothioneine or a similar expression refers to the amount of L-ergothioneine or a salt thereof as L-ergothioneine. Specifically, in the case of L-ergothioneine, the expression refers to the amount of L-ergothioneine; while in the case of a salt of L-ergothioneine, the expression refers to the value obtained by multiplying the number of moles of the salt by the molar weight of L-ergothioneine.

The term “quercetin” as used herein refers to quercetin, which is a compound belonging to the flavonoid group of polyphenols. In the present invention, the term “quercetin glycoside” refers to a glycoside of the quercetin. Specifically, it is a collective term for a series of compounds in which one or more sugar molecules are glycosidically linked to the hydroxy group at position 3 and/or the hydroxy group at position 4′ of quercetin. The quercetin glycoside may include one compound or two or more compounds. Examples of the sugar (monosaccharide) linked to quercetin include glucose, rhamnose, galactose, and glucuronic acids, with glucose and rhamnose being preferred. The sugar linked to quercetin may be modified (e.g., malonylated).

In one embodiment, the quercetin glycoside is preferably a compound in which one or more sugar molecules are glycosidically linked to the hydroxy group at position 3 of quercetin. A quercetin glycoside in which one or more sugar molecules are glycosidically linked to the hydroxy group at position 3 of quercetin is a compound represented by the following formula. In the following formula, (X)ₙ represents a glycan. X represents sugar (monosaccharide), and n is an integer of 1 or more.

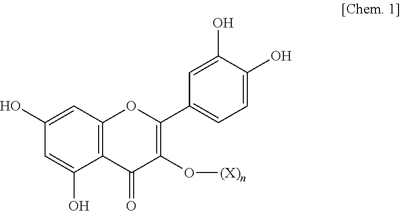

Examples of the sugar in a glycan, which is glycosidically linked to quercetin and represented by X, include glucose, rhamnose, galactose, and glucuronic acid, with glucose and rhamnose being preferred. In addition, n is not limited as long as it is 1 or more, but it is preferably 1 to 16, more preferably 1 to 8. When n is 2 or more, the X moiety may contain one type of sugar or multiple types of sugar. In other words, when n is 2 or more, (X)ₙ may be a glycan containing one type of sugar or a glycan containing multiple types of sugar.

The quercetin glycoside in the present invention encompasses a product obtained by treating an existing quercetin glycoside with an enzyme or the like for transglycosylation. The term “quercetin glycoside” as used in the present invention specifically encompasses rutin (quercetin 3-rutinoside), enzyme-treated rutin (enzyme-treated product of rutin), quercitrin (quercetin-3-O-rhamnoside), isoquercitrin (quercetin 3-glucoside), quercetin 3,4′-diglucoside, quercetin 4′-glucoside, quercetin 3-(6-malonylglucoside), hyperoside (quercetin 3-galactoside), quercetin-3-O-glucuronide, and the like. The enzyme-treated rutin is a quercetin glycoside in which an additional glycan is linked to a glucoside moiety of isoquercitrin. Preferred examples of the enzyme-treated rutin include a product in which a glycan containing one to seven glucose molecules is linked to isoquercitrin, the product being obtained by treating rutin with an enzyme to obtain isoquercitrin in which a rhamnose glycan moiety is removed and treating the resulting isoquercitrin with a glycosyltransferase. Preferred examples also include one mainly containing a mixture of such products. In one embodiment, quercetin or a glycoside thereof is preferably a quercetin glycoside, more preferably a quercetin glycoside in which one or more sugar molecules are glycosidically linked to the hydroxy group at position 3 of quercetin, still more preferably at least one selected from the group consisting of isoquercitrin, rutin, enzyme-treated rutin, and quercitrin, and yet still more preferably isoquercitrin or enzyme-treated rutin. The quercetin glycoside is particularly preferably a compound in which one to eight glucose molecules are glycosidically linked to the hydroxy group at position 3 of quercetin (e.g., isoquercitrin or a glycoside in which one to seven glucose molecules are linked to isoquercitrin), most preferably isoquercitrin.

In the present invention, quercetin or a glycoside thereof may be obtained from any source and by any process. Examples of plants known to contain a large amount of quercetin or a glycoside thereof include buckwheat, Japanese pagoda trees, caper, apples, tea, onions, grapes, broccoli, nalta jute, raspberries, cowberries, cranberries, prickly pears, leaf vegetables, and citrus fruits. Quercetin or a glycoside thereof can be obtained from these plants.

As long as the effect of the present invention is achieved, quercetin or a glycoside thereof for use in the present invention may be a naturally derived extract of any of the above plants in which the amount of quercetin or a glycoside thereof is increased by operations such as concentration and purification. For example, a concentrate or purified product of an extract containing quercetin or a glycoside thereof can be used. The extract can be concentrated or purified by a conventional method. In the present invention, purified or isolated quercetin or a glycoside thereof may be used. Chemically synthesized quercetin or a glycoside thereof can also be used.

The amount of quercetin or a glycoside thereof in terms of isoquercitrin in the composition, for example, is preferably 0.0001 wt % or more, more preferably 0.001 wt % or more, and may also be 50 wt % or less or 10 wt % or less, preferably 1 wt % or less, more preferably 0.1 wt % or less. In one embodiment, the amount of quercetin or a glycoside thereof in terms of isoquercitrin in the composition may be 0.0001 to 50 wt % or 0.0001 to 10 wt %, preferably 0.0001 to 1 wt %, more preferably 0.001 to 0.1 wt %.

The amount of quercetin or a glycoside thereof can be measured by high-performance liquid chromatography (HPLC).

Herein, the expression regarding the amount of quercetin or a glycoside thereof in terms of isoquercitrin or a similar expression refers to the amount of quercetin or a glycoside thereof as isoquercitrin. Specifically, in the case where a compound is isoquercitrin, the expression refers to the amount of isoquercitrin; while in the case where a compound is quercetin or a quercetin glycoside other than isoquercitrin, the expression refers to the value obtained by multiplying the number of moles of the compound by the molar weight of isoquercitrin.

In the present invention, the weight ratio of (A) L-ergothioneine or a salt thereof in terms of L-ergothioneine to (B) quercetin or a glycoside thereof in terms of isoquercitrin, i.e., (A) in terms of L-ergothioneine/(B) in terms of isoquercitrin, is preferably 0.01 or more, more preferably 0.02 or more, still more preferably 0.025 or more, and is preferably 1.2 or less, more preferably 1.1 or less, still more preferably 1 or less. When the weight ratio of (A) L-ergothioneine or a salt thereof in terms of L-ergothioneine to (B) quercetin or a glycoside thereof in terms of isoquercitrin ((A) in terms of L-ergothioneine/(B) in terms of isoquercitrin) is in the above range, the microorganism growth inhibitory action of quercetin or a glycoside thereof is effectively enhanced. In one embodiment, the weight ratio ((A) in terms of L-ergothioneine/(B) in terms of isoquercitrin) is preferably 0.01 to 1.2, more preferably 0.02 to 1.2, still more preferably 0.025 to 1.2, yet still more preferably 0.025 to 1.1, particularly preferably 0.025 to 1. Preferably, the composition of the present invention contains (A) L-ergothioneine or a salt thereof and (B) quercetin or a glycoside thereof at the above weight ratio.

The composition of the present invention contains (A) L-ergothioneine or a salt thereof and (B) quercetin or a glycoside thereof, so that microorganism growth therein is inhibited. The composition of the present invention is useful as a composition (preferably, an oral composition) with inhibited microorganism growth.

As an academic definition, the “microorganism” encompasses not only bacteria but also fungi (mushrooms, molds, yeast, etc.), viruses, microalgae, and the like, but the term “microorganism” as used herein refers to bacteria.

In the present invention, the microorganism is preferably Escherichia coli. A combination of L-ergothioneine or a salt thereof and quercetin or a glycoside thereof can exhibit an excellent growth inhibiting effect on Escherichia coli.

The composition of the present invention can be provided, for example, in the form of a food or beverage, a pharmaceutical product, a quasi-pharmaceutical product, feed, or the like. The composition of the present invention may be a food or beverage, a pharmaceutical product, a quasi-pharmaceutical product, feed, or the like by itself, or may be a material, preparation, or the like to be added thereto. In one embodiment, when the composition of the present invention is provided as a food or beverage, a pharmaceutical product, a quasi-pharmaceutical product, feed, or the like, the amount of L-ergothioneine or a salt thereof in terms of L-ergothioneine in the composition is preferably 0.0001 to 0.1 wt %, more preferably 0.001 to 0.01 wt %. The amount of quercetin or a glycoside thereof in terms of isoquercitrin in the composition is preferably 0.001 to 1 wt %, more preferably 0.01 to 0.1 wt %.

In one embodiment, preferably, the composition of the present invention is an oral composition. The oral composition may be a food or beverage, an oral pharmaceutical product, a quasi-pharmaceutical product, feed, or the like, preferably a food or beverage or an oral pharmaceutical product, more preferably a food or beverage.

In one embodiment, the composition of the present invention is useful as an oral composition with inhibited microorganism growth, preferably with inhibited Escherichia coli growth. In one embodiment, the composition of the present invention is useful as a product, such as a food or beverage, oral pharmaceutical product, oral quasi-pharmaceutical product, or feed, with inhibited microorganism growth.

The composition of the present invention can contain optional additives and optional components in addition to L-ergothioneine or a salt thereof and quercetin or a glycoside thereof, as long as the effect of the present invention is not impaired. Such additives and components may be selected depending on the form of the composition, for example. Those generally usable in, for example, food or beverages, pharmaceutical products, quasi-pharmaceutical products, and feed can be used. When the composition of the present invention is provided as a food or beverage, a pharmaceutical product, a quasi-pharmaceutical product, feed, or the like, such a product can be produced by any usual method.

The composition of the present invention may be in any form such as a solid (e.g., powder, granule, or tablet), liquid, or paste.

For example, when the composition of the present invention is provided as a food or beverage, components usable in food or beverages (e.g., food materials and optional food additives) can be added to L-ergothioneine or a salt thereof and quercetin or a glycoside thereof to provide various food or beverages. The food or beverage is not limited. Examples include general food or beverages, health foods, health drinks, foods with function claims, foods for specified health uses, health supplements, and foods for the sick. The health foods, foods with function claims, foods for specified health uses, health supplements, and the like can be used in various forms of preparations such as fine granules, tablets, granules, powders, capsules, chewable tablets, syrups, liquid agents, and liquid foods.

When the composition of the present invention is provided as a pharmaceutical product or a quasi-pharmaceutical product, for example, a pharmacologically acceptable carrier, an optional additive, and the like can be added to L-ergothioneine or a salt thereof and quercetin or a glycoside thereof to provide various dosage forms of pharmaceutical products or quasi-pharmaceutical products. Such a carrier, additive, or the like may be any pharmacologically acceptable one that can be used in pharmaceutical products or quasi-pharmaceutical products. Examples include excipients, binders, disintegrants, lubricants, antioxidants, and colorants. One or more of these can be used. Examples of the dosage form of the pharmaceutical product or the quasi-pharmaceutical product include oral administration and parenteral administration, with oral administration being preferred. When the composition of the present invention is provided as a pharmaceutical product or a quasi-pharmaceutical product, an oral pharmaceutical product or an oral quasi-pharmaceutical product is preferred. Examples of dosage forms for oral administration include liquids, tablets, powders, fine granules, granules, sugar-coated tablets, capsules, suspensions, emulsions, and chewable tablets. Examples of dosage forms for parenteral administration include injections and intravenous agents.

When the composition of the present invention is provided as feed, L-ergothioneine or a salt thereof and quercetin or a glycoside thereof are simply added to feed. The feed includes feed additives. Examples of the feed include livestock feed for animals such as cows, pigs, chickens, sheep, and horses; feed for small animals such as rabbits, rats, and mice; and pet food for animals such as dogs, cats, and birds.

When the composition of the present invention is provided as an oral composition, the composition of the present invention may be ingested by or administered to any subject (administration subject). The subject is preferably a human or non-human mammal, more preferably a human.

When the composition of the present invention is provided as an oral composition, the intake (administration amount) of the composition of the present invention is not limited. For example, in the case of a human (adult), the intake in terms of L-ergothioneine per day is preferably 2 to 50 mg, more preferably 5 to 25 mg, still more preferably 5 to 20 mg, particularly preferably 10 to 20 mg.

A combination of (A) L-ergothioneine or a salt thereof and (B) quercetin or a glycoside thereof can be used as an active ingredient for inhibiting microorganism growth. In one embodiment, the composition of the present invention can be suitably used, for example, for inhibiting microorganism growth. In one embodiment, the composition of the present invention may be a composition for inhibiting microorganism growth.

L-ergothioneine or a salt thereof and quercetin or a glycoside thereof are compounds that are found in natural products and food or beverages and that have been consumed. The composition of the present invention can be suitably used to inhibit microorganism growth in an oral composition, for example. In one embodiment, the composition of the present invention can be suitably used as a composition for inhibiting microorganism growth in an oral composition. The composition for inhibiting microorganism growth can also be referred to as “a microorganism growth inhibitor”. In one embodiment, the composition of the present invention can be suitably used as a composition for inhibiting Escherichia coli growth.

In one embodiment, the composition of the present invention can be used by being added to an oral composition, for example. When the composition of the present invention is used to inhibit microorganism growth in an oral composition, the composition of the present invention is simply added to the oral composition. In one embodiment, the composition of the present invention can be used as a food additive. The composition of the present invention may be added to any oral composition. Examples include food or beverages, oral pharmaceutical products, oral quasi-pharmaceutical products, and feed, with food or beverages being preferred. In one embodiment, when the composition of the present invention is used by being added as a composition for inhibiting microorganism growth to a food or beverage or the like, the total amount of (A) L-ergothioneine or a salt thereof and (B) quercetin or a glycoside thereof in the composition may be, for example, 0.01 to 99 wt %, preferably 0.1 to 90 wt %.

When the composition of the present invention is added to an oral composition to obtain the microorganism growth inhibiting effect, preferably, the composition is added to an oral composition such that the amount of L-ergothioneine or a salt thereof in terms of L-ergothioneine in the oral composition is preferably 0.00001 to 0.1 wt %, more preferably 0.0001 to 0.01 wt %. Preferably, quercetin or a glycoside thereof is added to an oral composition such that the amount of quercetin or a glycoside thereof in terms of isoquercitrin in the oral composition is preferably 0.001 to 1 wt %, more preferably 0.01 to 0.1 wt %. Preferably, the composition is added such that the amounts of L-ergothioneine or a salt thereof and quercetin or a glycoside thereof are as described above for obtaining the microorganism growth inhibiting effect.

### (Method of Inhibiting Microorganism Growth)

The present invention also encompasses a method of inhibiting microorganism growth in an oral composition, including adding (A) L-ergothioneine or a salt thereof and (B) quercetin or a glycoside thereof to an oral composition.

In the method of the present invention, (A) L-ergothioneine or a salt thereof, (B) quercetin or a glycoside thereof, and preferred embodiments thereof are as described above for the composition of the present invention.

For example, quercetin or a glycoside thereof is preferably at least one selected from the group consisting of isoquercitrin, rutin, enzyme-treated rutin, and quercitrin.

In the method of the present invention, the microorganism and its preferred embodiments are as described above, and the microorganism is preferably Escherichia coli. Preferably, the method of the present invention is a method of inhibiting Escherichia coli growth in an oral composition. The oral composition is not limited. Examples include the food or beverages, oral pharmaceutical products, oral quasi-pharmaceutical products, and feed, which are described above.

In the method of the present invention, preferably, (A) L-ergothioneine or a salt thereof and (B) quercetin or a glycoside thereof are added to an oral composition such that a weight ratio of (A) L-ergothioneine or a salt thereof in terms of L-ergothioneine to (B) quercetin or a glycoside thereof in terms of isoquercitrin, i.e., (A) in terms of L-ergothioneine/(B) in terms of isoquercitrin, is 0.01 or more. The weight ratio ((A) in terms of L-ergothioneine/(B) in terms of isoquercitrin) is more preferably 0.02 or more, more preferably 0.025 or more, and is also preferably 1.2 or less, more preferably 1.1 or less, still more preferably 1 or less. When the weight ratio of (A) L-ergothioneine or a salt thereof (in terms of L-ergothioneine) to (B) quercetin or a glycoside thereof (in terms of isoquercitrin) ((A) in terms of L-ergothioneine/(B) in terms of isoquercitrin) is in the above range, the microorganism growth inhibitory action of quercetin or a glycoside thereof is effectively enhanced. In one embodiment, the weight ratio ((A) in terms of L-ergothioneine/(B) in terms of isoquercitrin) is preferably 0.01 to 1.2, more preferably 0.02 to 1.2, still more preferably 0.025 to 1.2, yet still more preferably 0.025 to 1.1, particularly preferably 0.025 to 1. Preferably, (A) L-ergothioneine or a salt thereof and (B) quercetin or a glycoside thereof are added to an oral composition such that the weight ratio of (A) L-ergothioneine or a salt thereof (in terms of L-ergothioneine) to (B) quercetin or a glycoside thereof (in terms of isoquercitrin) ((A) in terms of L-ergothioneine/(B) in terms of isoquercitrin) in the oral composition is in the above range.

Preferably, L-ergothioneine or a salt thereof is added to an oral composition such that the amount of L-ergothioneine or a salt thereof in terms of L-ergothioneine in the oral composition is preferably 0.00001 to 0.1 wt %, more preferably 0.0001 to 0.01 wt %. Preferably, quercetin or a glycoside thereof is added to an oral composition such that the amount of quercetin or a glycoside thereof in terms of isoquercitrin in the oral composition is preferably 0.001 to 1 wt %, more preferably 0.01 to 0.1 wt %.

In the method of the present invention, (A) L-ergothioneine or a salt thereof and (B) quercetin or a glycoside thereof may be separately added to an oral composition, or a composition containing them may be added to an oral composition. For example, the composition of the present invention described above may be used to add (A) L-ergothioneine or a salt thereof and (B) quercetin or a glycoside thereof to an oral composition.

### (Agent for Enhancing Microorganism Growth Inhibitory Action of Quercetin or Glycoside Thereof)

The present invention also encompasses an agent for enhancing microorganism growth inhibitory action of quercetin or a glycoside thereof, the agent containing L-ergothioneine or a salt thereof. L-ergothioneine or a salt thereof can be used as an active ingredient for enhancing the microorganism growth inhibitory action of quercetin or a glycoside thereof.

L-ergothioneine or a salt thereof can be suitably used, for example, for enhancing Escherichia coli growth inhibitory action of quercetin or a glycoside thereof.

The present invention also encompasses use of (A) L-ergothioneine or a salt thereof and (B) quercetin or a glycoside thereof for producing a composition for inhibiting microorganism growth.

The present invention also encompasses use of L-ergothioneine or a salt thereof for enhancing microorganism growth inhibitory action of quercetin or a glycoside thereof.

The numerical range defined by the lower limit and the upper limit herein, i.e., “the lower limit to the upper limit”, includes the lower limit and the upper limit. For example, the range defined by “1 to 2” means 1 or more and 2 or less, with 1 and 2 being inclusive. Herein, the range may be any combination of any upper limit and any lower limit.

### EXAMPLES

The present invention is described in further detail below with reference to Examples, which do not limit the present invention.

### Example 1

The sterilizing (microorganism growth inhibiting) effect of L-ergothioneine (hereinafter, sometimes simply described as “ergothioneine”) and a quercetin glycoside was examined.

Isoquercitrin (quercetin 3-glucoside available from Cayman Chemical) was used as a quercetin glycoside.

Escherichia coli (Escherichia coli NBRC 3972) was used as a test bacterium.

### (Preparation of Test Bacterial Solution)

Escherichia coli was inoculated onto a standard agar medium and cultured at 30° C. to 35° C. for 24 hours. After culturing, the bacterial count was adjusted to 106 CFU/mL using saline, whereby a test bacterial solution was obtained.

### (Preparation of Test Sample)

Phosphate-buffered saline and ergothioneine were mixed to prepare a test sample 1 (ergothioneine concentration: 100 ppm (0.01 wt %)).

Phosphate-buffered saline and isoquercitrin were mixed to prepare a test sample 2 (isoquercitrin concentration: 100 ppm (0.01 wt %)).

Phosphate-buffered saline, ergothioneine, and isoquercitrin were mixed to prepare a test sample 3 (ergothioneine concentration: 2.5 ppm (0.00025 wt %), isoquercitrin concentration: 100 ppm (0.01 wt %)). Phosphate-buffered saline, ergothioneine, and isoquercitrin were mixed to prepare a test sample 4 (ergothioneine concentration: 100 ppm (0.01 wt %), isoquercitrin concentration: 100 ppm (0.01 wt %)).

Phosphate-buffered saline was used as control.

Table 1 shows the concentrations of (A) ergothioneine and (B) isoquercitrin and the weight ratio ((A)/(B)) of (A) ergothioneine to (B) isoquercitrin in each test sample.

TABLE 1

| | (A) Ergothioneine | (B) Isoquercitrin | Weight ratio |
|---|---|---|---|
| | (ppm) | (ppm) | (A)/(B) |
| Control | 0 | 0 | — |
| Test sample 1 | 100 | 0 | — |
| Test sample 2 | 0 | 100 | — |
| Test sample 3 | 2.5 | 100 | 0.025 |
| Test sample 4 | 100 | 100 | 1 |

### (Inoculation and Culturing of Test Bacterial Solution)

A test sample (5 mL) was inoculated with a 0.05 mL test bacterial solution and left standing at room temperature.

### (Viable Bacterial Counting)

One hour after inoculation, a 10-fold dilution of the test sample inoculated with the test bacterial solution was prepared in a soybean-casein digest liquid medium with lecithin and polysorbate 80. The resulting dilution containing Escherichia coli was inoculated onto a soybean-casein digest agar medium with lecithin and polysorbate 80 and cultured at 30° C. to 35° C. for three to five days. After culturing, the number of colonies formed was counted, and the viable bacterial count was calculated (n=3).

The sterilizing effect (%) was determined from the viable bacterial count (average of n=3) using the following formula.

Sterilizing effect (%)=100−100×(viable bacterial count in test sample)/(viable bacterial count in control)

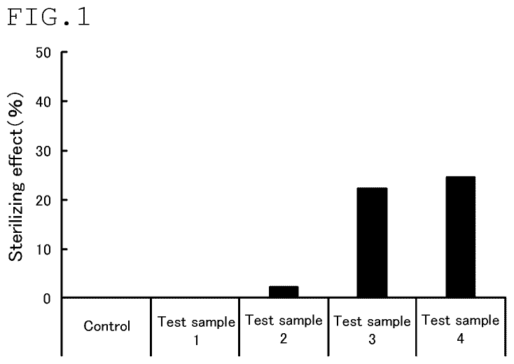

FIG. 1 shows the results.

FIG. 1 is a graph showing the sterilizing effect of L-ergothioneine (test sample 1), isoquercitrin (test sample 2), and a combination of L-ergothioneine and isoquercitrin (test samples 3 and 4). L-ergothioneine showed no sterilizing effect on Escherichia coli when used alone. Isoquercitrin inhibited Escherichia coli growth when used alone. The microorganism growth inhibiting effect on Escherichia coli obtained by the combination of L-ergothioneine and isoquercitrin was a synergetic effect that is significantly superior to an additive effect that is predicable from the effect obtainable from the use of each component alone. The results show that an excellent sterilizing effect on microorganisms, i.e., an excellent microorganism growth inhibiting effect, can be obtained with use of L-ergothioneine and isoquercitrin in combination.

1. A composition comprising:
(A) L-ergothioneine or a salt thereof; and
(B) quercetin or a glycoside thereof.

2. The composition according to claim 1,
wherein the (B) quercetin or a glycoside thereof is at least one selected from the group consisting of isoquercitrin, rutin, enzyme-treated rutin, and quercitrin.

3. The composition according to claim 1,
wherein a weight ratio of the (A) L-ergothioneine or a salt thereof in terms of L-ergothioneine to the (B) quercetin or a glycoside thereof in terms of isoquercitrin, i.e., (A) in terms of L-ergothioneine/(B) in terms of isoquercitrin, is 0.01 to 1.2.

4. The composition according to claim 1,
wherein the composition is a composition for inhibiting microorganism growth.

5. The composition according to claim 4,
wherein the composition is a composition for inhibiting microorganism growth in an oral composition.

6. The composition according to claim 4,
wherein the microorganism is Escherichia coli.

7. A method of inhibiting microorganism growth in an oral composition, comprising:
adding (A) L-ergothioneine or a salt thereof and (B) quercetin or a glycoside thereof to an oral composition.

8. The method according to claim 7,
wherein the (B) quercetin or a glycoside thereof is at least one selected from the group consisting of isoquercitrin, rutin, enzyme-treated rutin, and quercitrin.

9. The method according to claim 7,
wherein the (A) L-ergothioneine or a salt thereof and the (B) quercetin or a glycoside thereof are added to an oral composition such that a weight ratio of the (A) L-ergothioneine or a salt thereof in terms of L-ergothioneine to the (B) quercetin or a glycoside thereof in terms of isoquercitrin, i.e., (A) in terms of L-ergothioneine/(B) in terms of isoquercitrin, in the oral composition is 0.01 to 1.2.

10. The method according to claim 7,
wherein the microorganism is Escherichia coli.

11. An agent for enhancing microorganism growth inhibitory action of quercetin or a glycoside thereof, comprising:
L-ergothioneine or a salt thereof.
